(19) United States
(12) Patent Application Publication
(10) Pub. No.: US 2021/0228439 A1
(43) Pub. Date: Jul. 29, 2021

(54) Preparation of an internal massager and application thereof

(71) Applicant: Guangzhou Dawnlight Biotech Company, Guangzhou City, Guangdong Province (CN)

(72) Inventor: Yu Liu, Guangzhou City, Guangdong Province (CN)

(21) Appl. No.: 16/313,757

(22) PCT Filed: Mar. 14, 2018
(86) PCT No.: PCT/CN2018/078936

§ 371 Date: Dec. 27, 2018

(30) Foreign Application Priority Data
Jan. 2, 2018 (CN) 201810001614.X

(51) Int. Cl. A61H 21/00; A61H 15/00; A61H 39/04; A61L 2/025; A61L 2/10; A61L 2/04; A61L 2/18

(52) U.S. Cl. CPC A61H 21/00; A61H 15/0085; A61H 39/04; A61L 2/025; A61L 2/10; A61L 2101/34; A61L 2/18; A61H 2201/1207; A61H 2201/1253; A61H 2201/169; A61L 2202/24; A61L 2/04

(57) ABSTRACT

The internal massager consists of a massage ball, a rod, a protecting cover and a handle. The preparation of the internal massager includes: welding the massage ball to one end of the rod first, tightly connecting the other end of the rod with the protecting cover, and then welding the handle to the side of the protecting cover away from the rod, thereby to produce an internal massager; the internal massager massages and stimulates the throat, esophagus, and rectocolic surfaces, which promotes the local or systemic nervous-humoral reactions, improves the immune function of the body, and promotes the recovery of chronic diseases and physical weakness. The internal massager provided by the present invention has a simple preparation method, and the prepared internal massager has a wide range of application, significantly stimulatory effects in vivo, and therefore it has promoting effects on the recovery of chronic diseases and physical weakness.

### TECHNICAL FIELD

The present invention relates to the technical field of massage instruments, and in particular to preparation of an internal massager and application thereof.

### BACKGROUND

Traditional Chinese medicine theory believes that the acupuncture point massage on the human body may dredge the meridians and have effects for preventing diseases, curing diseases or health care. Huangdi Neijing reports: “Diseases with clinical manifestation as numbness of the body are resulted from panic and the meridian block, which can be treated with massage and wine.” This indicates that massage can be used to cure diseases. The modern medical research shows that massage mainly stimulates peripheral nerves and thereby promotes the blood, lymphatic circulations and the metabolism in tissues. It balances the functions between various tissues and organs, so that the metabolism in the body is improved. Clinical practice and animal experiments have also demonstrated that massage therapy has the effects in antiinflammation, defervescence, and immunity improvement, and therefore, massage therapy may enhance the body's resistance to diseases.

Luo Hong, a well-being practitioner in the Ming Dynasty, said in the Longevity Immortal Book: “Massage therapy can clean pores and harmonize qi and blood”. The expression harmonizing qi and blood here means regulation of qi and blood. This is the effect of Chinese massage. It uses a soft and light force applying at meridiansor acupuncture points of the body, balancing the whole body through conduction of meridians so as to reconcile the defensive Qi and blood nutrients, and thereby enhancing the health of the patient. Modern medical science believes that the mechanical stimulation of massage forms a combined action that converts the mechanical energy into heat energy, and one of the effects of Chinese massage is the increased temperature of local tissues, promoting capillary expansion, improving blood and lymph circulation, reducing blood viscosity, the peripheral vascular resistance and the burden on the heart, and therefore preventing or curing diseases.

Modern people are often living in a state of tension or nervousness which results in dysfunction of organs or even the whole body, manifested as neurosis, insomnia or forgetfulness. The Chinese massage works through meridians to improve the qi and blood circulate and the yin and yang balance. Therefore, after massage therapy the patient may feel muscle relaxation, joint flexibility, spiritual pleasure and fatigue elimination; which may result in a good sleep at night, an energetic feeling at the daytime, and finally a regular life, which in turn correct the imbalance among organs in the body. Moreover, massage therapy may also stimulate the appetite, increase the production of energy in the body, and improve the work efficiency. These effects of massage help the body to resist diseases. Therefore, a moderate massage in daily life may be beneficial to the physical and mental health. However, to date the Chinese massage is only implemented on the skin at different acupuncture points of the body, and the sensitivity of the skin to massage is poor compared to the internal surface or mucosa. Therefore, the stimulation caused by external massage on the skin is sometimes not strong enough and the therapeutic effects are not obvious.

The pharyngeal, esophageal and anorectal mucosal nerves are sensitive to physical stimulation, which accept either autonomic, sympathetic or parasympathetic regulations, and also produce local hormones or chemical factors that regulate the functions of the local or adjacent tissues and organs, such as the humoral regulatory factors intestinal 5-hydroxytryptamine and gastrin. Therefore, stimulation of the stated mucosa may improve gastrointestinal functions, promote food digestion and intestinal nutrition metaplasia, and enhance physical fitness and disease resistance. At the same time, stimulation of pharyngeal-esophageal and anorectal mucosal nerves may feedback to the central nervous system, resulting in the regulation of the whole body. The feedback of body activities such as eating, swallowing and egestion to the central nervous system may achieve the effects such as relaxation and sedation of the body.

Therefore, internal massager stimulating the nervous activity of the pharyngeal-esophageal and anorectal mucosa, just as the responses to eating, swallowing and egestion, may achieve the effects such as body relaxation, sedation, promotion of nutrition metaplasia, and improvement of disease resistance of the body. Based on these facts, the present invention provides preparation of an internal massager and the application thereof.

### SUMMARY

The purpose of the present invention is to provide a preparation technique of an internal massager and the application thereof in order to overcome the drawbacks of the prior art conventional massage.

Provided is the preparation of an internal massager, wherein the internal massager consists of a massage ball, a rod, a protecting cover and a handle; preparation of the internal massager includes the following steps: welding the massage ball to one end of the rod first, connecting the other end of the rod to the protecting cover, and then welding the handle to the side of the protecting cover away from the rod, so as to produce an internal massager.

Preferably, the number of the massage balls is 1 through 5, the diameter of the massage ball is from 0.5 to 3 in cm, and the massage balls are arranged at appropriate intervals on the rod if there are a plurality of massage balls.

Preferably, the material of the massage ball, the rod, the protecting cover and the handle is any of metal, gold-plated metal, silver-plated metal, tin-plated metal, non-metal material, gold-plated non-metal material, silver-plated non-metal material, or tin-plated non-metal material; the metal is any of stainless steel, copper, platinum, or iron, the non-metal material is any of plastic, rubber, glass, jade, ceramic, or plasticized products, and the massage ball may also contain a magnetic material.

Preferably, the handle is solid or is internally installed with a battery or a motor.

The present invention further provides the application of an internal massager, wherein the internal massager massages and stimulates the throat, esophagus, anorectal and intestinal surfaces, which promotes the local or systemic nervous-humoral reactions, regulates the immune functions of the body, and improves the recovery of chronic diseases and physical weakness.

Preferably, the chronic disease is a physical disease that has not been cured for more than 15 days, or still exists for more than 1 month with repeated attacks or even continuous deterioration, and the chronic disease comprises cancer, tuberculosis, Alzheimer's disease, hypertension, and diabetes.

Preferably, the physical weakness includes low body immunity, debilitated physical strength and energy, sub-health state, or a pathological change in the body that has not been cured for more than 15 days or that exists for more than 1 month; the physical weakness comprises weakness after illness, weakness in the elderly, and physical weakness due to the side effects of tumor chemoradiotherapy or other treatments.

Preferably, the method of using the internal massager comprises the steps of: selecting either physical or chemical sterilization method for sterilization prior to the use of the internal massager, and placing the internal massager on the internal surface of the body as stated above, massaging for 10 to 300 seconds each time, 1 to 6 times per day.

Preferably, the physical sterilization method is any of: ultraviolet radiation sterilization, ultrasonic sterilization, high-temperature sterilization or stewing sterilization, and the chemical sterilization method is alcohol sterilization at the concentration of 75% by w/v.

Preferably, if uncomfortable reactions occur during the operation of the internal massager, the massage should be stopped, and edible grease or lubricant agents may be applied on the internal massager to comfort the massage.

Compared to the prior art, conventional massage, the beneficial effects of the present invention are as follows.

1. The preparation of the internal massager is simple in method and wide in material source, and it may be metal or non-metal, and may be plated with gold, silver or tin film on the surface. The internal massager may be prepared as several types and may be applied to a variety of internal areas of the body, so to expand the scope of its application. Moreover, the massage ball may also contain a magnetic material to provide an effect of magnetic therapy.

2. It is demonstrated by animal experiments that the internal massager method provided by the present invention inhibited tumor growth through pharyngeal-esophageal internal massage, and it may have a nervous-humoral reaction after use. Therefore, the internal massager may be used to assist traditional Chinese and Western medicines in the treatment of chronic diseases or may be used to promote the recovery of physical weakness with remarkable effects. The long-term use of the internal massager provided by the present invention for internal massage, such as pharyngeal-esophageal massage, may also improve the body immunity, and thereby it may have a broad application prospect in health care.

3. The internal massager technique makes up for the drawbacks of conventional massage and acupuncture that the external body surface massage is not sensitive enough, while the internal massager may be directly applied internally, which increases the stimulatory effects and improves the treatment of diseases.

### BRIEF DESCRIPTION OF THE DRAWINGS

FIG. 1 is a schematic view illustrating the structure of a single-ball manual internal massager prepared according to the first embodiment of the present invention;

FIG. 2 is a schematic view illustrating the structure of a multi-ball manual internal massager prepared according to the second embodiment of the present invention;

FIG. 3 is a schematic view illustrating the structure of a single-ball electric internal massager prepared according to the third embodiment of the present invention;

FIG. 4 is a schematic view illustrating the structure of a multi-ball electric internal massager prepared according to the fourth embodiment of the present invention;

FIG. 5 is a graph illustrating the change in tumor volume in an experiment in which the pharyngeal-esophagus massage inhibited tumor growth; and

FIG. 6 is a histogram illustrating the change in serum 5-hydroxytryptamine in an experiment in which the pharyngeal-esophagus massage promotes a nervous-humoral reaction.

In the figures, 1 denotes the massage ball, 2 denotes the rod, 3 denotes the protecting cover, 4 denotes the handle, and 5-HT is an abbreviation of 5-hydroxytryptamine.

### DESCRIPTION OF THE EMBODIMENTS

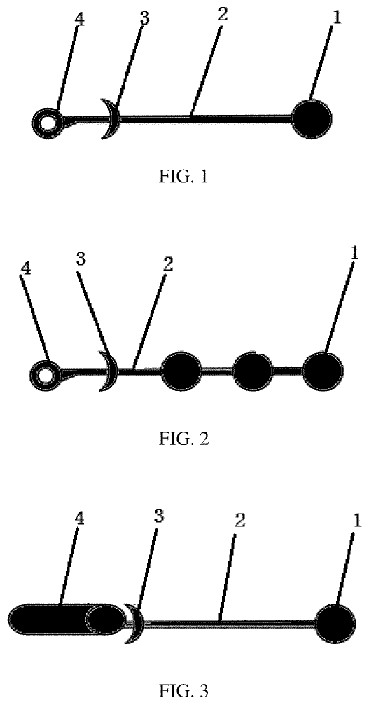

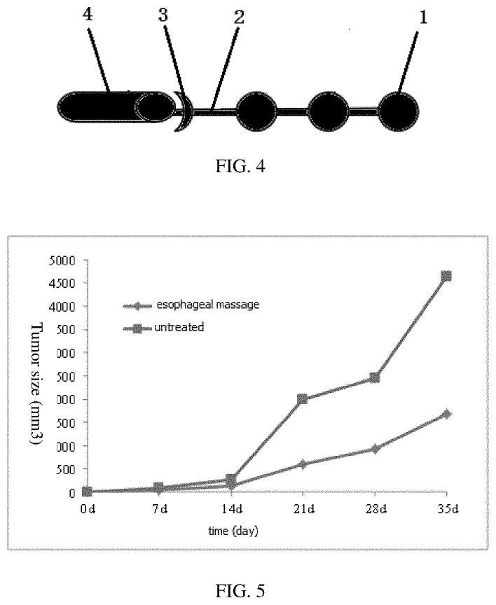

With reference to FIGS. 1-6, the present invention is further illustrated below in conjunction with specific embodiments.

### A First Embodiment

The present invention provides the preparation of an internal massager, wherein the internal massager consists of a massage ball 1, a rod 2, a protecting cover 3 and a handle 4. The preparation of the internal massager includes the following steps: welding the massage ball 1 to one end of the rod 2 first, tightly connecting the other end of the rod 2 with the protecting cover 3, and then welding the handle 4 to the side of the protecting cover 3 away from the rod 2, thereby to produce an internal massager. The number of the massage ball 1 is 1, the diameter of the massage ball is 0.5 cm, the material of the massage ball 1 is stainless steel, and the material of the rod 2, the protecting cover 3 and the handle 4 is copper.

### A Second Embodiment

The present invention provides the preparation of an internal massager, wherein the internal massager consists of a massage ball 1, a rod 2, a protecting cover 3 and a handle 4. The preparation of the internal massager includes the following steps: welding the massage ball 1 to one end of the rod 2 first, tightly connecting the other end of the rod 2 with the protecting cover 3, and then welding the handle 4 to the side of the protecting cover 3 away from the rod 2, thereby to produce an internal massager. The number of the massage ball 1 is 3, the massage balls 1 are arranged at appropriate intervals on the rod 2, the diameter of the massage balls is 1.5 cm, the material of the massage ball 1 is silver-plated copper, and the material of the rod 2, the protecting cover 3 and the handle 4 is plastic.

### A Third Embodiment

The present invention provides the preparation of an internal massager, wherein the internal massager consists of a massage ball 1, a rod 2, a protecting cover 3 and a handle 4. The preparation of the internal massager includes the following steps: connecting the massage ball 1 to one end of the rod 2 first, tightly connecting the other end of the rod 2 with the protecting cover 3, then connecting the handle 4 to the side of the protecting cover 3 away from the rod 2, and installing a battery inside the handle 4, thereby to produce an electric internal massager. The number of the massage ball 1 is 1, the diameter of the massage ball 1 is 2 cm, the material of the massage ball 1 is jade, and the material of the rod 2, the protecting cover 3 and the handle 4 is iron.

### A Fourth Embodiment

The present invention provides the preparation of an internal massager, wherein the internal massager consists of a massage ball 1, a rod 2, a protecting cover 3 and a handle 4. The preparation of the internal massager includes the following steps: connecting the massage ball 1 to one end of the rod 2 first, tightly connecting the other end of the rod 2 with the protecting cover 3, and then connecting the handle 4 to the side of the protecting cover 3 away from the rod 2, thereby to produce an internal massager. The number of the massage ball 1 is 1, the diameter of the massage ball is 3 cm, the material of the massage ball 1 is glass, and the material of the rod 2, the protecting cover 3 and the handle 4 is tin-plated copper.

### A Fifth Embodiment

The present invention provides the preparation of an internal massager, wherein the internal massager consists of a massage ball 1, a rod 2, a protecting cover 3 and a handle 4. The preparation of the internal massager includes the following steps: connecting the massage ball 1 to one end of the rod 2 first, tightly connecting the other end of the rod 2 with the protecting cover 3, and then connecting the handle 4 to the side of the protecting cover 3 away from the rod 2, thereby to produce an internal massager. The number of the massage ball 1 is 1, the diameter of the massage ball is 1.5 cm, the material of the massage ball 1 is ceramic which is fire-formed by a common method, and the material of the rod 2, the protecting cover 3 and the handle 4 is plastic.

### A Sixth Embodiment

The present invention provides the preparation of an internal massager, wherein the internal massager consists of a massage ball 1, a rod 2, a protecting cover 3 and a handle 4. The preparation of the internal massager includes the following steps: connecting the massage ball 1 to one end of the rod 2 first, tightly connecting the other end of the rod 2 with the protecting cover 3, and then connecting the handle 4 to the side of the protecting cover 3 away from the rod 2, thereby to produce an internal massager. The number of the massage ball 1 is 1, the diameter of the massage ball is 2 cm, the material of the massage ball 1 is jade, the jade contains magnetic material and is prepared by a common method, and the material of the rod 2, the protecting cover 3 and the handle 4 is plastic.

Animal Experiment:

1) The Experiment of Mouse Tumor Inhibition

Two groups of mice were selected and between the two groups there was no significant difference in age, weight, sex and health status. S180 tumor cells were subcutaneously transplanted into each mouse of the two groups in the area of the right armpit. The first group of mice were treated with pharyngeal-esophagus massage using a single-ball manual massager in size of a gavage needle prepared based on the first embodiment. The massage was performed for 10 seconds each time, once a day. The massage treatment was carried out for 35 days. The second group of mice were not treated. The tumor size was measured in the two groups of mice, and the average tumor size was calculated for each group of mice to plot a graph illustrating the change in tumor volume on the 0, 7^{th}, 14^{th}, 21^{st}, 28^{th}, and 35^{th} day since the experiment.

The graph of FIG. 5 illustrating the change in tumor volume shows that the average tumor volume of the mice subjected to pharyngeal-esophagus massage with the single-ball manual massager prepared according to the first embodiment is significantly smaller than that of the untreated mice. Moreover, the tumor size change on the 28th and 35th days is statistically significant P<0.05, which indicates that the internal massager prepared by the stated preparation method of the present invention can inhibit tumor growth by pharyngeal-esophagus massage.

2) The Experiment of Nervous-Humoral Reaction in Mice

Two groups of mice were selected and between the two groups there was no significant difference in age, weight, sex and health status. The first group of mice were subjected to pharyngeal-esophagus massage with the multi-ball manual internal massager in size of a gavage needle prepared based on the second embodiment. Pharyngeal-esophagus massage was performed for 10 seconds, once in each mouse. The second group of mice were not treated. The serum 5-hydroxytryptamine levels in the two groups of mice were measured at the time-points 0, 1 h, 8 hs, 16 hs and 24 hs after the treatment, and a histogram is plotted.

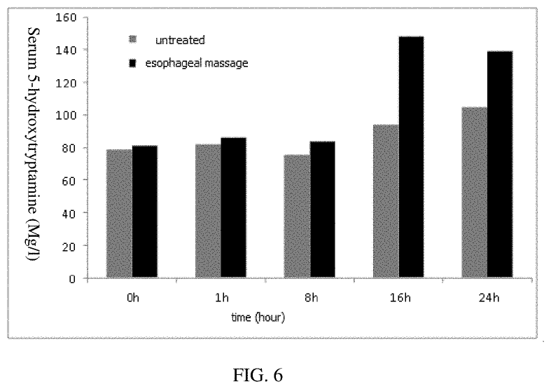

The histogram of FIG. 6 shows that the serum 5-hydroxytryptamine level of the mice subjected to pharyngeal-esophagus massage once with a multi-ball manual internal massager prepared according to the second embodiment was significantly higher than that of the mice without massage treatment, which indicates that pharyngeal-esophagus massage with the internal massager prepared based on the preparation method of the present invention caused a nervous-humoral reaction in the body.

The foregoing descriptions are merely preferred to the embodiments of the present invention, but the scope of protection of the present invention is not limited thereto. Any equivalent replacements or changes made by those skilled in the art within the technical scope disclosed by the present invention according to the technical solution of the present invention and the inventive concept thereof should all fall within the scope of protection of the present invention.

What claimed is:

1. Preparation of an internal massager, wherein the internal massager consists of a massage ball (1), a rod (2), a protecting cover (3) and a handle (4); the preparation of the internal massager includes the following steps: welding the massage ball (1) to one end of the rod (2) first, tightly connecting the other end of the rod (2) with the protecting cover (3), and then welding the handle (4) to the side of the protecting cover (3) away from the rod (2), thereby to produce an internal massager.

2. The preparation of an internal massager according to claim 1, wherein the number of the massage balls (1) is from 1 to 5, the diameter of the massage ball (1) is from 0.5 to 3 cm, and the massage balls (1) are arranged at appropriate intervals on the rod (2) if a multiball-massager is produced.

3. The preparation of an internal massager according to claim 1, wherein the material of the massage ball (1), the rod (2), the protecting cover (3) and the handle (4) is any of metal, gold-plated metal, silver-plated metal, tin-plated metal, non-metal material, gold-plated non-metal material, silver-plated non-metal material, or tin-plated non-metal material, the metal is any of stainless steel, copper, platinum, or iron, the non-metal material is any of plastic, rubber, glass, jade, ceramic, or plasticized products, and the massage ball (1) may also contains a magnetic material.

4. The preparation of an internal massager according to claim 1, wherein the handle (4) is solid or is internally installed with a battery or a motor.

5. Application of an internal massager, wherein the internal massager massages and stimulates the throat, esophagus, and rectocolic surfaces, which promotes the local or systemic nervous-humoral reaction, regulates the immune function of the body, and has a promoting effect on the recovery of chronic diseases and physical weakness.

6. The application of an internal massager according to claim 5, wherein the chronic disease is a physical disease that has not been cured for more than 15 days or still exists for more than 1 month with repeated attacks or even continuous deterioration, and the chronic disease includes cancer, tuberculosis, Alzheimer's disease, hypertension, and diabetes.

7. The application of an internal massager according to claim 5, wherein the physical weakness includes low immunity, debilitated physical strength and energy, and sub-health or a pathological change in the body that has not been cured for more than 15 days or still exists for more than 1 month; the physical weakness is the weakness after illness, or weakness in the elderly, or weakness due to the side effects of tumor chemoradiotherapy or treatment of other diseases.

8. The application of an internal massager according to claim 5, wherein the method of using the internal massager comprises the following steps: selecting either physical or chemical sterilization methods for sterilization of the internal massager before use, then placing the internal massager in the massaging area for 10 to 300 seconds each time, 1 to 6 times per day.

9. The application of an internal massager according to claim 8, wherein the physical sterilization is any of ultraviolet radiation, ultrasonic sterilization, high-temperature or stewing sterilization, and the chemical sterilization is alcohol sterilization at a mass concentration of 75%.

10. The application of an internal massager according to claim 8, wherein if an uncomfortable reaction occurs during the operation of the massage stimulation, the massage should be stopped and an edible grease or lubricant substance is applied on the internal massager for a continuing massage.
